(19) United States
(12) Patent Application Publication
(10) Pub. No.: US 2023/0129139 A1
(43) Pub. Date: Apr. 27, 2023

(54) SYSTEM AND METHOD FOR PROVIDING A MEDICATION AT A MEDICAL DEVICE

(71) Applicant: B. Braun Melsungen AG, Melsungen (DE)

(72) Inventors: Horst Schmoll, Guxhagen (DE); Michael Duesterhus, Hessisch Lichtenau (DE); Christoph Erlen, Kassel (DE)

(21) Appl. No.: 17/970,631

(22) Filed: Oct. 21, 2022

(30) Foreign Application Priority Data
Oct. 22, 2021 (DE) 10 2021 127 476.1

(51) Int. Cl. G16H 20/10; G16H 10/60

(52) U.S. Cl. CPC G16H 20/10; G16H 10/60

(57) ABSTRACT

A drug management system and a method for providing a specific medication at a medical device. The system includes a central drug database and at least one external, decentral medical device. A drug prescription can be input preferably manually via an input interface, and an appropriate drug dataset can be retrieved for the medical device and supplied to the medical device. The central drug database includes a plurality of drug datasets for the medical device. The central drug database is configured to match and validate the drug prescription with the plurality of drug datasets and prepare the prescription so as to only implement the drug dataset corresponding to the drug prescription from the central drug database in the medical device during use of the medical device.

### CROSS-REFERENCE TO RELATED APPLICATION

This application claims priority under 35 U.S.C. § 119 to German Application No. 10 2021 127 476.1, filed Oct. 22, 2021, the content of which is incorporated by reference herein in its entirety.

### FIELD

The present disclosure relates to a medication providing system with a central drug database and at least one medical (medication-administering) device, in particular an infusion pump, syringe pump, etc., and to a method for providing a specific medication at at least one such medical (medication-administering) device.

### BACKGROUND

In operation of a hospital it is of relevance that a number of (patient treatment) data, such as, for instance a (patient-specific) medication, is provided so that a user, for instance, a nurse, physician, caring person, etc. can administer drugs safely to a patient. This data provision can, as is generally known, be performed by means of a medicament library/drug library/database or a dose error reduction software. In other words, this is a database which includes inter alia information about various drugs. This is, for instance, the type of drug itself (effect, fields of application, side effects, etc.), the drug concentration in the carrier substance, and dosing parameters including relevant limits.

In current implementations of medicament libraries/drug libraries/databases the respective library/database is stored locally (decentrally) on/in the medical device, such as an infusion pump or the control thereof. In the case of updates of the contents of the library/database, therefore, all medical devices/infusion pumps in a hospital have to be updated before the new data are available in the medical device/in the infusion pump for use. It is common that possible updates are only performed at the corresponding medical device which has to be used subsequently. Also, a waiver of updates for time reasons and the use of outdated data is not uncommon. Likewise, it would basically be possible, comparable with update methods in telecommunication devices, to update the individual databases of the individual medical devices by WLAN, LAN, or Bluetooth and an appropriate app, which, however, necessitates a correspondingly expensive updating network and good connection situations.

Specifically the kind of implementation (individually) performed locally at the medical device is conceived such that it is performed as an offline scenario in which generic medical devices (pumps) require no access to the IT infrastructure. Despite the nature of such an offline scenario each online therapy configuration, in the following also called auto-programming, is processed completely on/by the medical device (pump) itself. In other words this means that a medication (or drug parameter) in accordance with a current auto-programming prescription is electronically processed locally on the generic medical device (e.g. infusion pump).

The afore-mentioned “auto-programming” enables generally the build-up of a “closed loop system” in order to implement a safe medication workflow and to avoid device/pump-induced undesired medication events in that the medication data are checked twice. In this process, much programming work at a medical device, such as an infusion pump, and expenditure of time for documentation is saved.

The process of such a medication workflow starts basically with the prescription/drug prescription/auto-programming prescription. The physician inputs the drug prescription in a patient data management system. The information contained in this prescription is used for the mixing/preparing/providing of the drug, and based on the information an appropriate label is produced in a further process step. Medication data contained in the information is transferred to the site of the drug admixture. In parallel/during the preparation of the drug, barcoding with a patient-individual drug information and a correspondingly established patient ID takes place. On beginning of the therapy/start of the treatment the medication process step starts with the scanning of the patient ID encoded in a barcode, for instance, in an armband of the patient, the scanning of the device/pump identification, the scanning of the label of the corresponding drug container, and optionally the ID of the physician or the nurse. The medication data are then transferred to the device/the pump if the prescription/drug prescription/auto-programming prescription matches with the scanning data. The data are displayed at the medical device/the pump, and the clinician can finally validate the medication data. Following a menu configuration/menu confirmation by the clinician the drug administration/infusion can be started. Therefore, the auto-programming serves primarily to increase medication safety.

Due to a real time operation, for instance, of infusion pumps in clinical practice it is in many cases not possible to perform an update of the drug database in due time. Consequently, many devices of this type, such as infusion pumps, therefore use drug databases with outdated drug datasets. Another reason for the slow updating of drug databases is that it is only possible to update the complete drug database on the medical device/the infusion pump. Since medical devices/infusion pumps are devices with limited resources embedded in a system, the medicament library/drug database may be limited with respect to the number of drugs supported and the associated drug datasets. In other words, the storage capacity of the individual medical device/pump is limited, and depending on the number of drug datasets, not all drug datasets may be stored on each medical device/each infusion pump.

From the state of the art drug management systems are already known. For instance, U.S. Pat. No. 8,478,604 B2 discloses a system and a method for the use of drugs with a closed loop, comprising the selection of a drug to be prescribed to a patient, based on patient information such as laboratory results, radiologic results, and patient allergies, practices of healthcare industry, site-specific guidelines for patient care, and drug information. The selected drug is prescribed on a non-examined prescription which is then transcribed. Transcribing comprises the performing of a plurality of counterchecks of the prescription in real time with patient information, practices of healthcare industry, and drug information so as to establish a verified prescription. After the transcription, the suited output method for the prescription is determined and output. The drug output is, after confirmation of the correct patient, the correct drug, the correct dose, the correct way, and the correct time, administered by the physician in charge. The entire described process of drug use is monitored continuously in real time. The monitored information is transmitted to the prescribing, transcribing, outputting, and managing parts of the system.

Further, EP 3 217 304 A1 describes an infusion pump system, comprising
at least one pump adapted to be attached to a patient and to cause infusion of a drug into the patient's body,
a storing unit adapted to store at least a plurality of therapy data, and
information about particularities of therapies already carried out or currently running,
an input unit adapted to enable a selection of specific therapy data representing a specific therapy from said plurality of therapy data.

EP 1 702 285 A1 describes a system and a method for monitoring, managing, and controlling the drug output from a central site. A central computer indicates drug prescriptions and current drug managements for a health facility. The central computer examines the drug output by means of a database with guidelines for drug administration including guidelines for the interaction of drugs with other drugs and with patient conditions and renders information about incompatibilities found. A clinician at the central site may adapt the parameters for the drug administration in response to incompatibilities found and may communicate with a caring person at the treatment site so as to give decision support.

### SUMMARY

In view of this state of the art, it is an object of the present disclosure to provide a system which remedies or at least improves the disadvantages of the state of the art and in which it is possible to always provide current data prior to the beginning of a therapy in order to care for a patient safely and as good as possible.

The object of the present disclosure is solved by a system with a central drug database and at least one external, decentral medical device, in particular an infusion pump, wherein the system is in particular provided and designed such that a drug prescription can be input preferably manually via an input interface and an appropriate drug dataset for the at least one medical device can be retrieved and supplied to it, wherein the central drug database comprises a plurality of drug datasets for the at least one medical device and the central drug database is provided and designed to match and validate the drug prescription with the plurality of drug datasets and to prepare it so as to transfer, during use of the at least one medical device, only the drug dataset corresponding to the drug prescription from the central drug database and the prepared drug prescription in the at least one medical device.

In other words, the present disclosure is intended to provide a central drug database within a hospital IT infrastructure, in which individual drug datasets are retrievable or can be retrieved from all and/or individual medical devices connected, in particular infusion pumps. This is due to the fact that, in many hospitals all over the world, infusion pumps are connected to an IT network.

In this respect it is of advantage if the centralized drug database can maintain the site of a particular medical device, in particular an infusion pump, within a hospital organization and can provide the correct drug dataset for this site. In this case, for instance, by means of the centralized drug database an automatic differentiation may be made between an intensive care unit and a pediatric care unit when providing drug datasets. Moreover, site-specific drug datasets minimize the selection process at the medical device, in particular the infusion pump, itself. This means in other words that the site is preselected for the drug selection and rate for facilitating operation. Accordingly, the drug selection and rate is, for instance, adapted to children or adults and/or intensive patients, depending on the site. In other words, the site information enables only a preselection/pre-configuration corresponding to the site for facilitated operation, but may be adapted at any time to a drug dataset which does not correspond to the site.

Furthermore, the subject matter of the present disclosure has the advantage that the access to the central drug database offers information, in particular infusion data, always with current drug datasets. Accordingly, no more outdated drug datasets are used in the drug databases and updates of drug datasets are immediately available. This means further that updates need not be performed at each medical device individually, but only once in the central drug database which provides the appropriate drug dataset for the corresponding medical device(s) as needed.

It is preferred if the at least one medical device is provided and designed to directly communicate and interact with the central drug database after the input of the drug prescription.

It is of advantage if the at least one medical device is provided as the input interface. In other words, the input of the drug prescription takes place by a nurse or a physician/clinician directly at the at least one and/or the respective medical device.

In other words, a local operation at the at least one medical device is provided primarily in an online operation. In an online operation, the user/the nurse types the name of the drug in accordance with the drug prescription into the at least one medical device, the drug prescription is processed by the central drug database, and the correct drug dataset for this drug is, in correspondence with the drug prescription, provided by the central drug database and transmitted to the medical device. Accordingly, the access to the central drug database provides medical information, in particular infusions, always with current drug datasets. Offline operation is also possible, but this will not be dealt with here in detail.

It is preferred if the drug dataset provided by the central drug database can be used to update the drug dataset of the local drug database as a fallback dataset for offline operation. This means in other words that at least the one selected drug dataset can be stored as a snapshot on the medical device so as to be able to resort to it in the case of a missing network connection/in offline operation.

Moreover, it is of advantage if, in an offline operation, the drug dataset of the medical device can be used for the therapy configuration.

It is of advantage if the system comprises a patient data management system and an interface, wherein the interface is provided and designed to communicate and to interact with the patient data management system, with the central drug database, and with the at least one medical device.

In other words, it is preferred if in an online operation and/or also remote operation and/or auto-programming any therapy information provided by the patient data management system for at least one particular medical device is compared with the central drug database and only in the case of a positive matching the drug prescription and the appropriate drug dataset are sent/transferred to the at least one medical device. Otherwise, an error description is additionally sent to the at least one medical device, which is directly visualized/displayed on the medical device for the user/the nurse.

For the case of an error description, which may be based on a failure of communication, it is possible for the user/clinician to make the necessary input manually directly at the medical device unless data stored locally in the medical device exist. For the case of an error description which is based on a contradiction between the data delivered and technical possibilities/specifications of the medical device, it is possible for the user/clinician to refuse the corresponding dataset or to input an alternative. Accordingly the user can actively make input at the medical device at any time, for instance, to be able to use higher rates not predetermined by the dataset.

In other words it is preferred if the interface between the patient data management system and the central drug database is provided for transmitting/transferring/forwarding the drug prescription, and it is preferred if the interface between the central drug database and the at least one medical device is provided to transmit/to transfer/to forward the appropriate drug dataset from the central drug database and the drug prescription appropriately prepared by the central drug database to the at least one medical device.

It is preferred if the patient data management system is provided as the input interface. This means if the information of the drug prescription is transmitted directly into the at least one medical device.

It is of advantage if the interface is a gateway. With respect to the present disclosure the gateway serves primarily as a switch for forwarding data.

Alternatively, the patient data management system is provided and designed such that the drug prescription can be input and to transmit the drug prescription input to the central drug database via the interface.

Advantageously, the central drug database is provided and designed to obtain at least one drug prescription via the interface/the gateway, to match and to validate the at least one drug prescription with the plurality of drug datasets of the central drug database, and to select an appropriate drug dataset to supply the appropriately selected drug dataset to the at least one medical device via the interface/the gateway.

It is preferred if the central drug database is provided and designed to process the drug prescription before the at least one drug prescription is provided at the at least one medical device.

Advantageously the system is provided and designed to send and visually display an error description to/at the at least one medical device in the case of an unsuccessful validation.

It is of advantage if the drug dataset corresponding to the drug prescription can be requested and/or transmitted independently of the at least one medical device.

The adding of specific integrity information for obtaining/ensuring the integrity of the drug dataset sent to the medical device is preferred.

Preferably, the central drug database is provided and designed to centrally update the plurality of drug datasets and/or at least one drug dataset.

Furthermore, the present disclosure relates to a method for providing a specific medication at at least one medical device, in particular at least one infusion pump, comprising the following steps:

(a) inputting a drug prescription via an input interface, wherein the input is performed in the at least one medical device or in a patient data management system;

(b) transmitting the drug prescription from the at least one medical device to a central drug database or from the patient data management system via an interface/a gateway to the central drug database;

(c) matching and validating the drug prescription with the central drug database and selecting an appropriate drug dataset;

(d) returning the one appropriate drug dataset to the at least one medical device (3) or transferring the one appropriate drug dataset via the interface/the gateway to the at least one medical device; and

(e) implementing the one appropriate drug dataset at the at least one medical device.

In other words, the disclosure provides an improved method for communication and interaction between a central drug database, for instance, within a hospital with local medical devices, for instance, infusion pumps so as to match the selection of the locally available drug datasets with the respective medical range of application and to ensure that drug-specific datasets in the local medical devices are always on a current status.

It is preferred if the input of the drug prescription in a patient data management system and the transmission via an interface take place in the IHE-/HL7 standard, wherein this means a group of international standards for the exchange of data between organizations in healthcare and their computer systems.

It is of advantage if the appropriate drug dataset is retrievable prior to the beginning of a therapy.

It is of advantage if a preselection of the drug dataset is made on the basis of the site of the at least one medical device.

To sum up, medical devices, in particular infusion pumps, with drug datasets which are stored locally on the pump have been provided so far. Based on the subject matter of the present disclosure the usage of the central drug database is here based on the fact that it is divided into a plurality of drug datasets which are provided to be requested in real time. Furthermore, auto-programming requests are not matched and possibly validated at the medical device with a local drug database, but preprocessed by a central drug database.

Accordingly, the central drug database offers a collection of drug datasets. Each drug dataset may be requested and/or transmitted independently of the pump. This enables a medical device to request the most recent version of a drug dataset in the ongoing operation from the central drug database. Auto-programming requests and/or prescriptions are not transmitted directly to the pump, but are first matched and validated with the appropriate central drug dataset. If the auto-programming request is invalid, i.e., no appropriate drug dataset is stored in the central drug database, the error description instead of the auto-programming prescription is sent to the at least one medical device. This enables the same feedback on the pump as it had been the case with a local input and/or a local auto-programming input/search.

This kind of implementation is conceived such that it can be executed as an online scenario in that the at least one medical device, in particular an infusion pump, requires access to the IT infrastructure to obtain the complete functional support. Drug-specific information in an online therapy configuration/auto-programming are first processed in the central drug database before an appropriately modified prescription is transmissible/is transmitted to the at least one medical device.

In order to obtain the integrity of the data sent to the medical device, specific integrity information is added. In the case of a failure (drug, therapy parameter, site, etc.) the automatic auto-programming prescription is refused toward the initiator of the request while for the use at the device side a specific alert/error message with the description of the problem is displayed/visualized. In this case a drug prescription is input and processed locally at the device side.

### BRIEF DESCRIPTION OF THE DRAWING FIGURES

FIG. 1 is a representation for illustrating a system construction with local management in accordance with the state of the art.

FIG. 2 is a representation for illustrating a system construction with remote management according to the state of the art.

FIG. 3 is a representation for illustrating a system construction with local management in accordance with an embodiment of the foregoing disclosure.

FIG. 4 is a representation for illustrating a system construction with remote management in accordance with an embodiment of the foregoing disclosure.

### DETAILED DESCRIPTION

In the following, embodiments of the present disclosure will be described on the basis of the associated Figures.

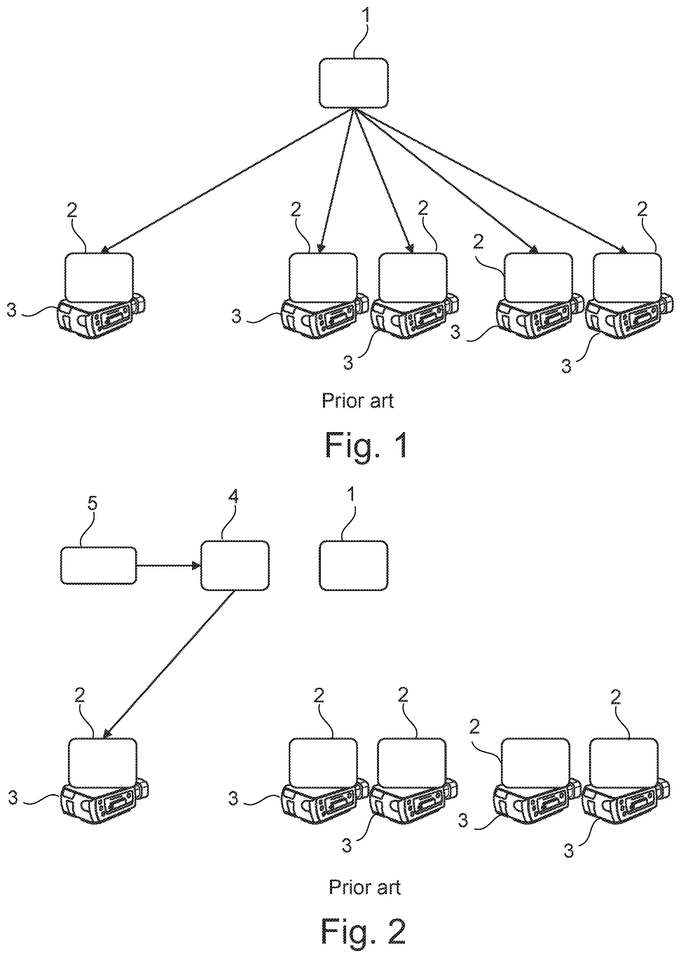

FIG. 1 is a representation for illustrating a system construction with local management in accordance with the state of the art. FIG. 1 shows a central drug database 1 and a plurality of medical devices 3. Via the central drug database 1 the drug datasets of the central drug database 1 are pushed to every single medical device 3 of the plurality of medical devices 3. This means that a (onetime) local storage 2 of the drug datasets of the central database 1 takes place on every single medical device 3. In other words, the drug datasets of the central drug database 1 were embedded in each of the plurality of medical devices 3.

The local storage 2 on every single medical device 3 can be accessed by a clinician/a nurse directly at the corresponding medical device 3. A clinician/a nurse chooses the drug required in correspondence with a drug prescription from the local storage 2/local drug database for local administration.

FIG. 2 is a representation for illustrating a system construction with remote management in accordance with the state of the art. FIG. 2 shows a central drug database 1 and a plurality of medical devices 3. On each of the plurality of medical devices 3 a local storage 2 is disposed. Furthermore, an interface 4 and a patient data management system 5 are illustrated in FIG. 2.

The clinician/the nurse inputs in the patient data management system 5 a drug prescription/a description which is transmitted to one of the corresponding medical devices 3 via the interface 4. Subsequently, it is inquired on the corresponding medical device 3 whether the drug of the drug prescription is contained in the local storage 2. If the drug is present, it is used, if the drug is not present in the local storage 2, an error message/error description is output directly at the corresponding medical device 3.

In the state of the art, possible updates of drug datasets first have to be performed prior to use by the central drug database 1 on the corresponding medical device. Accordingly, it is not possible for the clinician/the nurse to access the most current drug datasets in real time. Moreover, the storage capacity on the individual medical devices 3 is limited, so that possibly not all drug datasets can be stored. In the state of the art no matching and/or validating with the central drug database 1 is provided.

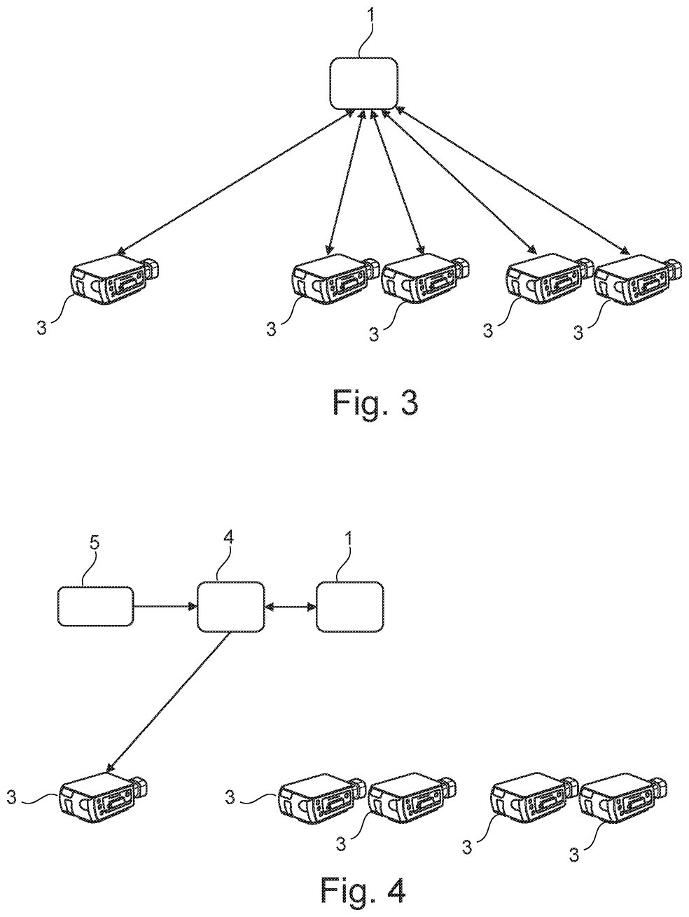

FIG. 3 is a representation for illustrating a system construction with local management in accordance with an embodiment of the foregoing disclosure. FIG. 3 shows in accordance with FIG. 1 a central drug database 1 and a plurality of medical devices 3. Each medical device 3 is in direct contact with the central drug database 1. This means that, if a clinician/a nurse inputs a drug prescription at the medical device 3, the drug datasets corresponding to the drug prescription are fetched/retrieved directly from the central drug database 1 and implemented in the corresponding medical device 3. Thus, only the required drug datasets for administering the drug are provided in real time and in the most current version.

FIG. 4 is a representation for illustrating a system construction with remote management in accordance with an embodiment of the foregoing disclosure. FIG. 4 shows a central drug database 1, a plurality of medical devices 3, an interface 4, and a patient data management system 5.

The clinician/the nurse inputs in the patient data management system 5 a drug prescription/a description which is transmitted to the central drug database 1 via the interface 4. In the central drug database the drug prescription is matched and validated with the drug datasets stored in the central drug database 1, and the drug dataset corresponding to the drug prescription is selected. The one appropriate drug dataset and the drug prescription are returned to the at least one medical device 3 via the interface 4, and the appropriate drug dataset is implemented at the at least one medical device 3.

In a case in which no drug dataset corresponding to the drug prescription is present in the central drug database 1, an error message/error description is output/visualized directly at the corresponding medical device 3. In this case the drug prescription must be input in the infusion pump manually. Furthermore, it is provided that the appropriately selected drug dataset is storable for therapy as a “snapshot” on the corresponding medical device 3 for the case of an offline operation.

In this manner, more drug data sets are available than can be stored locally in a medical device 3. Another advantage is the request of appropriate drug datasets in real time and the fact that the drug prescriptions are already prepared centrally in the central drug database 1.

What is claimed:

1. A system comprising:
a central drug database;
at least one medical device that is external and decentralized; and
an input interface,
the system configured to:
receive a drug prescription that is input into the input interface;
retrieve a drug dataset corresponding to the drug prescription; and
supply the drug dataset to the at least one medical device,
the central drug database comprising a plurality of drug datasets for the at least one medical device, and
the central drug database configured to match and validate the drug prescription with the plurality of drug datasets and to prepare the drug prescription so as to transfer, during use of the at least one medical device, only the drug dataset corresponding to the drug prescription from the central drug database and the prepared drug prescription into the at least one medical device.

2. The system according to claim 1, wherein the at least one medical device is provided and designed to communicate and interact directly with the central drug database after the input of the drug prescription.

3. The system according to claim 1, wherein the system comprises a patient data management system and an interface, wherein the interface is provided and designed to communicate and to interact with the patient data management system, with the central drug database, and with the at least one medical device, and wherein
the patient data management system is provided and designed to prepare the drug prescription and to transfer it to the central drug database via the interface.

4. The system according to claim 1, wherein the central drug database is provided and designed to process the drug prescription before the at least one drug prescription is provided at the at least one medical device.

5. The system according to claim 1, wherein the system is provided and designed to send and visually display an error description to/at the at least one medical device in the case of an unsuccessful validation.

6. The system according to claim 1, wherein the system is configured to request and/or transfer the drug dataset corresponding to the drug prescription independently of the at least one medical device.

7. The system according to claim 1, wherein the central drug database is configured to centrally update the plurality of drug datasets.

8. The system according to claim 1, wherein the central drug database is configured to provide site-specific datasets.

9. A method for providing a specific medication at at least one medical device that is external and decentral, the method comprising the steps of:
(a) inputting a drug prescription via an input interface, wherein an input is performed in the at least one medical device or in a patient data management system;
(b) transmitting the drug prescription from the at least one medical device to a central drug database or from the patient data management system via an interface to the central drug database;
(c) matching and validating the drug prescription with the central drug database and selecting an appropriate drug dataset;
(d) returning the appropriate drug dataset to the at least one medical device or transferring the appropriate drug dataset via the interface to the at least one medical device; and
(e) implementing the appropriate drug dataset at the at least one medical device.

10. The method according to claim 9, wherein the input of the drug prescription and transferring the appropriate drug dataset via the interface are performed in the IHE-/HL7 standard.

11. The method according to claim 10, wherein the appropriate drug dataset is retrievable prior to a therapy.

12. The method according to claim 9, wherein a preselection of the appropriate drug dataset is made based on a site of the at least one medical device.
